(19) United States
(12) Patent Application Publication
(10) Pub. No.: US 2023/0121396 A1
(43) Pub. Date: Apr. 20, 2023

(54) RESPIRATOR WITH AIR SCRUBBING SYSTEM

(71) Applicant: Falcon Power, LLC, Titusville, FL (US)

(72) Inventors: Harley C. McDonald, Merritt Island, FL (US); James L. Bailey, Titusville, FL (US)

(21) Appl. No.: 17/914,629

(22) PCT Filed: Mar. 26, 2021
(86) PCT No.: PCT/US2021/024381

§ 371 Date: Sep. 26, 2022

Related U.S. Application Data
(60) Provisional application No. 63/001,142, filed on Mar. 27, 2020.
(60) Provisional application No. 63/005,109, filed on Apr. 3, 2020.
(60) Provisional application No. 63/028,515, filed on May 21, 2020.
(60) Provisional application No. 63/104,271, filed on Oct. 22, 2020.

(51) Int. Cl. F24F 3/167; A62B 23/02; A62B 7/10; A62B 9/00; A62B 18/00; F24F 8/22; F24F 5/00

(52) U.S. Cl. CPC F24F 3/167; A62B 23/02; A62B 7/10; A62B 9/003; A62B 18/006; F24F 8/22; F24F 5/0042

(57) ABSTRACT

An air delivery system for delivering a pathogen-free airflow can include an air intake manifold, a sterilizer unit with a tank and at least one ultraviolet (UV) unit, a cooling unit with at least one cooling manifold and at least one heat exchange unit, and a user output. The air intake manifold, the tank, the at least one cooling manifold, and a user output can be in fluid communication in that general order. The at least one ultraviolet (UV) unit can be mounted within the tank, with the at least one UV unit configured to generate ultraviolet light to promote killing of pathogens. The at least one heat exchange unit is configured to cool the outbound air flow to a temperature suitable for breathing by a user and to heat an incoming air flow in the air intake manifold.

### RELATED APPLICATIONS

The present application claims domestic priority under 35 U.S.C. § 119(e) to U.S. Provisional Application Ser. No. 63/104,271, filed Oct. 22, 2020, and titled “RESPIRATOR WITH AIR SCRUBBING SYSTEM;” U.S. Provisional Application Ser. No. 63/028,515, filed May 21, 2020, and titled “RESPIRATOR WITH AIR SCRUBBING SYSTEM;” U.S. Provisional Application Ser. No. 63/005,109, filed Apr. 3, 2020, and titled “RESPIRATOR WITH AIR SCRUBBING SYSTEM;” and U.S. Provisional Application Ser. No. 63/001,142, filed Mar. 27, 2020, and titled “RESPIRATOR WITH AIR SCRUBBING SYSTEM.” The contents of each of U.S. Provisional Application Ser. Nos. 63/104,271; 63/028,515; 63/005,109; and 63/001,142 are hereby incorporated by reference thereto.

### BACKGROUND

A respirator is a device designed to protect the wearer from inhaling hazardous atmospheres, including particulate matter such as dusts and airborne microorganisms, as well as hazardous fumes, vapors, and gases. There are two main categories: the air-purifying respirator in which respirable air is obtained by filtering a contaminated atmosphere, and the air-supplied respirator in which an alternate supply of breathable air is delivered. Within each category, different techniques are employed to reduce or eliminate noxious airborne contaminants.

### DRAWINGS

The Detailed Description is described with reference to the accompanying figures.

FIG. 1 is a schematic view of a wearable air delivery system, in accordance with an embodiment of the present disclosure.

FIG. 2 is a schematic view of the air pump used in conjunction with the wearable air delivery system of FIG. 1.

FIG. 3 is a schematic view of the air heater and the UV unit used in conjunction with the wearable air delivery system of FIG. 1.

FIGS. 4A-4C are schematic views of the cooling unit, including its components, used in conjunction with the wearable air delivery system of FIG. 1.

FIG. 5 is a schematic view of the air mask used in conjunction with the wearable air delivery system of FIG. 1.

FIG. 6 is a schematic view of the power source used in conjunction with the wearable air delivery system of FIG. 1.

FIG. 7 is a schematic view of the computer used in conjunction with the wearable air delivery system of FIG. 1.

FIG. 8 is an isometric view of a sterilized air delivery system, in accordance with an embodiment of the present disclosure.

FIG. 9 is an isometric view of an air intake manifold, in conjunction with related portions of the sterilized air delivery system shown in FIG. 8.

FIG. 10 is an isometric view of the Peltier cooling modules of the sterilized air delivery system shown in FIG. 8.

FIG. 11 is an isometric view of one of the cooling manifolds of the sterilized air delivery system shown in FIG. 8.

FIG. 12 is an isometric view of an end cap and related components used in the sterilized air delivery system shown in FIG. 8.

FIG. 13 is an isometric view of a sterilized air delivery system, in accordance with an embodiment of the present disclosure.

FIG. 14 is a cut-away view of the sterilized air delivery system shown in FIG. 13.

FIG. 15 is a schematic view of a room air delivery system, in accordance with an embodiment of the present disclosure.

FIG. 16 is a schematic view of a recirculating room air delivery system, in accordance with an embodiment of the present disclosure.

FIG. 17 is a top, isometric view of a sterilized air delivery system, in accordance with an embodiment of the present disclosure.

FIG. 18 is a top, isometric view of the heating and cooling components of the sterilized air delivery system shown in FIG. 17.

FIG. 19 is a top, isometric view of a sterilized air delivery system shown in FIG. 18, without a top portion of the housing associated therewith.

FIG. 20 is a right, top, isometric view of the sterilized air delivery system shown in FIG. 19.

FIG. 21 is a left, top, isometric view of sterilized air delivery system shown in FIG. 19.

FIG. 22 is a left, top, isometric view of the sterilized air delivery system shown in FIG. 21, with a removable watt section displaced to expose the heat exchange components therebehind.

FIG. 23 is a sectional view along line 23-23 in FIG. 18 of the heating and cooling components of the sterilized air delivery system.

FIG. 24 is a sectional view along line 24-24 in FIG. 18 of the heating and cooling components of the sterilized air delivery system.

FIG. 25 is a sectional view along line 25-25 in FIG. 18 of the heating and cooling components of the sterilized air delivery system.

FIG. 26 is a sectional view along line 26-26 in FIG. 18 of the heating and cooling components of the sterilized air delivery system.

FIG. 27 is a side, isometric view of the primary heat exchanger system of the heating and cooling components of the sterilized air delivery system shown in FIG. 18.

FIG. 28 is a side, isometric view of the secondary cooling unit of the heating and cooling components of the sterilized air delivery system shown in FIG. 18.

FIG. 29 is a side, cut-away view of an example ultraviolet (UV) unit of the sterilized air delivery system shown in FIG. 18.

FIG. 30 is a schematic view of a sterilized air delivery system, in accordance with an embodiment of the present disclosure.

### DETAILED DESCRIPTION

Aspects of the disclosure are described more fully hereinafter with reference to the accompanying drawings, which form a part hereof, and which show, by way of illustration, example features. The features can, however, be embodied in many different forms and should not be construed as limited to the combinations set forth herein; rather, these combinations are provided so that this disclosure will be thorough and complete, and will fully convey the scope.

### Overview

In certain use environments, such as ones where airborne pathogens (e.g., bacteria and/or viruses) are the primary concern, the ability of the respirator to either filter out or kill airborne microorganisms is paramount. Also, in such environments, other types of particulates (e.g., dust) or hazardous fumes, vapors, and/or gases may present a generally minimal risk. Thus, it can be beneficial to have a respirator system that can focus on eliminating airborne pathogens (i.e., sterilizing the air) by treating the air flow therethrough, prior to reaching an end user.

In an embodiment, the present air delivery system can provide a solution to the purification of breathable air for, for example, first responders and healthcare workers in a form of a lightweight portable backpack unit, a fanny pack unit, or chest pack. Air can be first run through a mild filter to remove dust particles, translated through copper or other conductive metal tubes or conduits, where it is heated with temperature-controlled Peltier heat exchange elements to a temperature sufficient to kill or at least breakdown a lipid shell/shield of any airborne pathogens (e.g., approximately 150 degrees or more). It can then enter a small hermetically sealed copper tank where it circulates and is irradiated with, for example, two UV LEDs (250 to 300 nm wavelength) at up to 20 watts, each facing each other on opposite ends of the tank, rendering the pathogens inert. In an embodiment, the sterilized return air can be cooled by a cooling side of the same Peltier heat exchange elements, prior to delivery to an end user.

In an embodiment, the present air delivery system (e.g., a respirator) can treat the air in such a manner that kills any airborne pathogens (e.g., germs or other contagions) in the air supply before delivery of any such air to the end user (e.g., a person or an animal). In an embodiment, the air delivery system can first heat the air to a temperature sufficient to kill most airborne pathogens (e.g., bacteria, funguses, protozoa, and/or viruses) and then cool the air to a temperature (e.g., room temperature) compatible for breathing, prior to delivery to an end user. In some embodiments, the air delivery system can heat the air to a temperature of at least 140° F.-150° F. In some embodiments, the air delivery system can heat the air to a temperature in a range of 165° F.-250° F.

In an embodiment, the air delivery system can expose the air to a sufficient amount of ultraviolet (UV) radiation to kill and/or neutralize most, if not all, airborne pathogens, prior to delivery of that air to an end user. In an embodiment, the air may be subjected to 5-10 watts of UV radiation. In an embodiment, short wavelength UV light (known as UV-C), with a wavelength between about 200 nm and 300 nm, may be used. UV-C has been referred to as “germicidal UV.” A UV dosage, a product of UV light intensity and exposure time, can be measured in microjoules per square centimeter, or equivalently as microwatt seconds per square centimeter (μW·s/cm2). For example, dosages for a 90% kill of most bacteria and viruses range from 2,000 to 8,000 μW·s/cm2. Larger parasites such as cryptosporidium can require a lower dose for inactivation. In some embodiments, the air delivery system can use a combination of high-heat (e.g., 140° F. or more) and UV dosing to kill any airborne pathogens, prior to delivering the air to the end user.

In some embodiments, two or more wavelength of UV radiation may be provided to help kill or neutralize a greater variety of microbes or pathogens (e.g., viruses, bacteria, etc.). In some embodiments, the UV dosing can further be performed in the warm-up and/or cool-down sections of the system to increase the UV exposure time. In an embodiment, the UV dosing may occur throughout the entire system (e.g., UV units may be placed at various locations in the system).

Further, the air delivery system may include particulate filters and/or other types of filtration mechanisms to eliminate other forms of, for example, respiratory irritants and/or hazardous airborne materials. Such air delivery units may incorporate one or more mechanical filters (e.g., a simple dust filter) and/or chemical cartridges. However, it is to be understood, in an embodiment, that the air delivery system may only incorporate mechanisms (e.g., heat and/or UV) to kill airborne pathogens, along with any pumping and delivery components. That is, the goal of the air delivery system may be simply to provide an airflow of pathogen-free air to an end user. In embodiment, the air delivery system may further include a simple dust/particulate filter (e.g., not necessarily a HEPA-grade filter).

In one embodiment, the air delivery system may be a wearable respirator device for a given end user. In an embodiment, the wearable respirator device may be battery powered or coupled to an electric power supply (e.g., an AC power outlet). The wearable respirator device may, for example, be worn by first responders or other medical personnel. Different sizes of the air delivery system can be made available. The user output delivery mode can take other forms, beyond an end-user air mask, that are configured to deliver an air flow that is pathogen-free to an end user. In an embodiment, the air delivery system may be a larger system configured to deliver pathogen-free air to a cabin of a vehicle (e.g., car, truck, or military transport vehicle); one or more rooms in a building, tent, or other enclosed structure for use by one or more end users located in such a room or vehicle cabin; or the environment (e.g., purify air being exhausted from a room where a contagion may otherwise exist). Thus, the air delivery system may be scaled, as desired, based on a given need.

### Example Embodiments

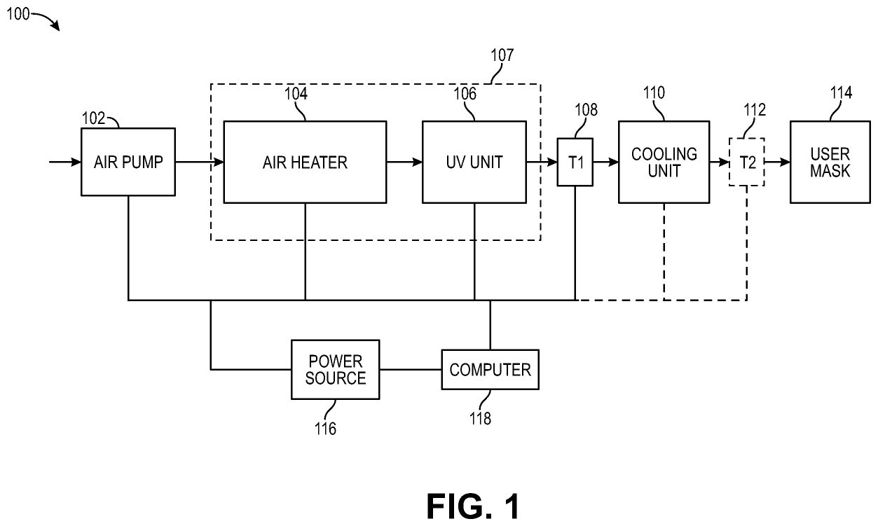

FIGS. 1-7 illustrate a wearable air delivery system 100, in accordance with an example embodiment of the present disclosure. The wearable air delivery system 100 can be a wearable respirator configured to be used by a specific end user (e.g., a person such as a first responder or other medical personnel), capable of delivering pathogen-free air to an end user. The wearable air delivery system 100 can include an air pump 102, an air heater 104, a UV unit 106 (e.g., for delivering UV-C to kill germs), a first temperature sensor 108 (i.e., to confirm sufficient heating (e.g., 140° F. or greater)), at least one cooling unit 110, an optional second temperature sensor 112 (e.g., to confirm sufficient cooling for user breathing (e.g., to room temperature)), a user mask 114, a power source 116, and a computer 118. The air pump 102, the air heater 104, the UV unit 106, the first air temperature sensor 108, the at least one cooling unit 110, the optional second temperature sensor 112, and the user mask 114 can be connected within the flow path of the air and, where appropriate (e.g., non-sensor components), can contain and direct the flow of the air. In an embodiment, the wearable air delivery system 100 can carried by end user (e.g., as part of a backpack unit, fanny pack unit, or chest pack), and the non-pump components thereof may weigh two pounds or less or one pound or less. In an embodiment, the wearable air delivery system 100 can weigh 2-3 pounds or less, including the pump. In an embodiment, the wearable air delivery system 100, in its entirety, can be 200 square inches (sq. in.) or less in volume or 100 sq. in. or less in volume (e.g., 2″×5″×10″).

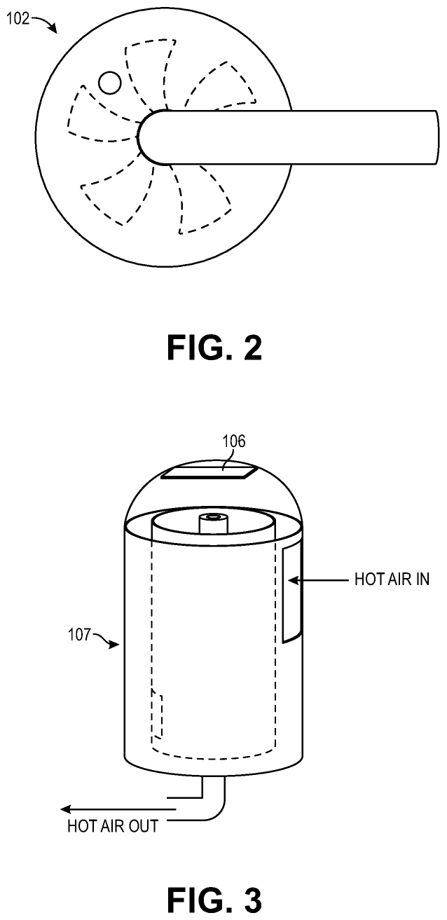

The air pump 102 is shown in FIG. 2. In an embodiment, the air pump 102 may include a fan. The air pump 102 is configured to draw the incoming air flow into the air delivery system 100. In an embodiment, the air pump 102 can be a CPAP (continuous positive airway pressure) type pump to maintain, ultimately, a positive purified air pressure within the user mask 114. In an embodiment, the air pump 102 can be a small electric pump, similar in size and power as a pump used for inflating and/or deflating air mattresses.

The air heater 104 (e.g., a least Peltier heat-exchange module 132 (also used as part of the cooling unit 110); at least one infrared diode; and/or a set of resistance coils) and the UV unit 106 (e.g., at least one UV light emitting diode (LED)) may, as illustrated, be part of a sterilization unit 107 configured to kill pathogens in the air flow, as shown in FIG. 3. In an embodiment, in a case of when the pathogen is a virus, heating the air can break down the outer lipid/fat shield around the virus, and then the UV-C irradiation can break up the DNA of the virus within, for example, less than 500 ms (milliseconds) (e.g., within 200 ms), thereby rendering the virus inert. It is to be understood that the air heater 104 and the UV unit 106 can be separate units used in tandem (e.g., heat first, then UV irradiate) or as part of a single unit. The first temperature sensor 108 can be proximate the air heater 104 (e.g., either in the air heater 104 or positioned near an exit therefrom). In an embodiment, the first temperature sensor 108 can be used to help confirm whether the air is heated to a temperature sufficient to kill airborne pathogens or at least breakdown any lipid outer shell thereof. In an embodiment the temperature sensors 108, 118 can be, by way of example, a thermocouple, RTD (resistance temperature detectors), thermistors, or non-contact IR (infrared) detector.

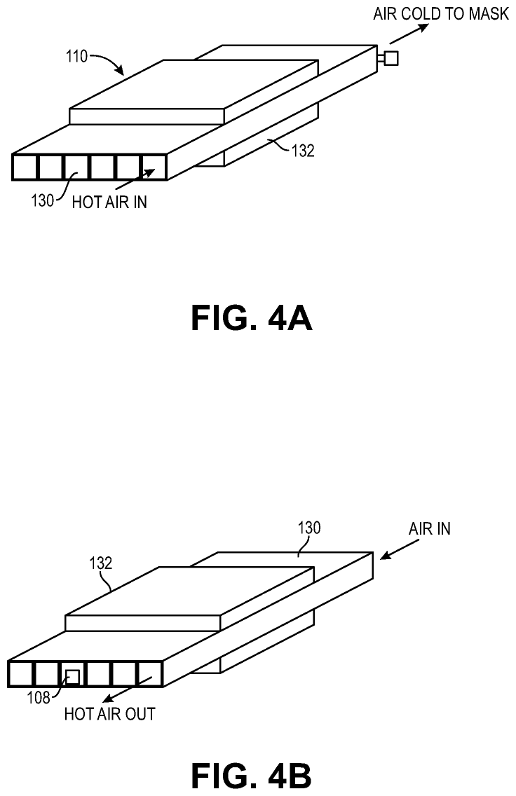

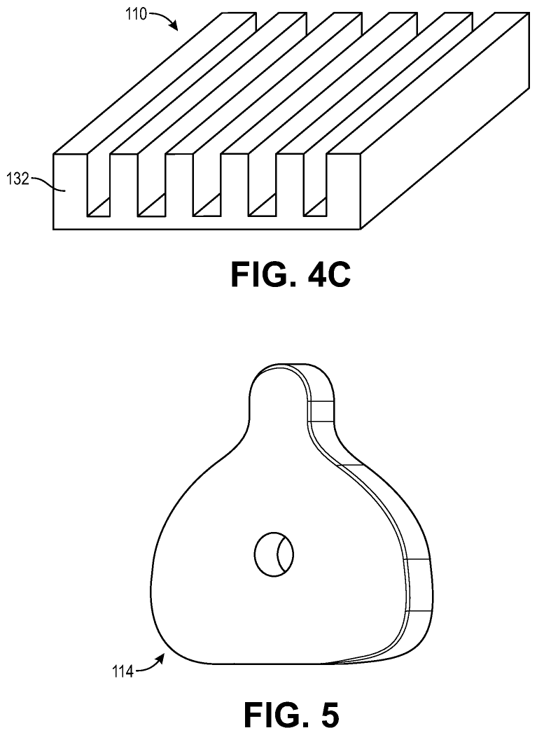

FIGS. 4A-4C illustrate the cooling units 110 (e.g., a combination of at least one Peltier heat exchange module 132 and at least one cooling manifold 130) is further shown in FIGS. 4A-4C. A Peltier cooler, heater, or thermoelectric heat pump is a solid-state active heat pump which transfers heat from one side of the device to the other, with consumption of electrical energy, depending on the direction of the current. The cooling unit 110 may, in one capacity, act as a cooling manifold to direct the airflow away from the air heater 104 toward an end user and simultaneously heat an air intake (shown in greater detail in the second embodiment). The optional second temperature sensor 112 can be located in the air flow at a position proximate the exit of the cooling unit 110 (e.g., either interior or exterior thereof) and used to help confirm whether sufficient cooling of the air flow for use by an end user has been achieved. In an embodiment, the one or more Peltier modules, in a second capacity, can be used to heat the incoming air to a temperature (e.g., 140-150° C. or more) sufficient to kill airborne pathogens or at least breakdown a lipid outer shell thereof (e.g., in the case of a virus), while aiding in cooling the air exiting toward an end user. In such an instance, the one or more Peltier modules can be configured as the air heater 104 for the system 100. In some embodiments, the one or more Peltier modules can be used simply to preheat the incoming air, which can then be fully heated to a sterilization temperature by a further air heater 104 (e.g., an infrared (IR) diode).

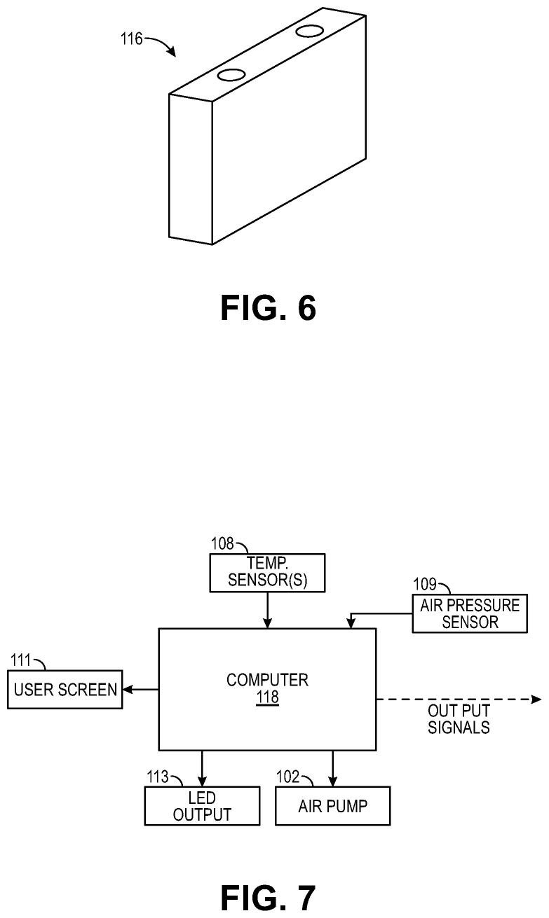

The user mask 114 (e.g., a CPAP mask), capable of fluid connection with the outbound airflow from the cooling unit 110, is shown in FIG. 5. The power source 116 (e.g., a battery pack (e.g., a plurality of lithium polymer batteries) or a connection to an outside power source, such as a plug-in connection) is shown in FIG. 6. The computer 118 is shown in FIG. 7, along with its electronic connections with other appropriate elements in the system. The computer 118 can receive inputs, for example, from one or more temperature sensors 108 or one or more air pressure sensors 109 and can provide outputs (e.g., control signals) to the air pump 102, a user screen 111, an LED output (e.g., for PWM), and one or more control signals to regulate, for example, the hot and cold temperatures and/or the level of air flow through the system (not numbered). The computer 118 is thereby configured to control operation of, for example, the air pump 102, the air heater 104, the UV unit 106, and/or the Peltier cooling component (where present) of a given cooling unit 110.

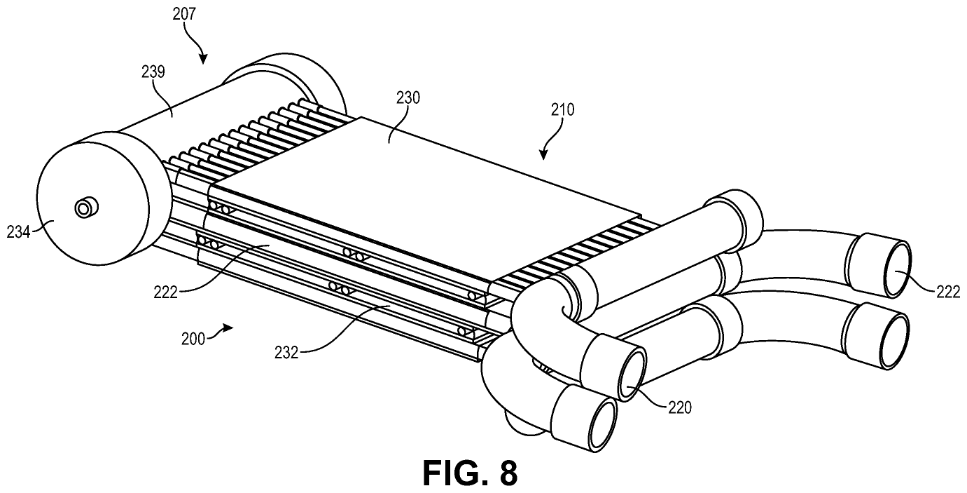

FIGS. 8-12 illustrate an embodiment of a sterilized air delivery system 200, in accordance with the present disclosure. Similar numbered parts for the sterilized air delivery system 200 can be expected to function and be constructed in a similar manner as for the wearable air delivery system 100, except as otherwise noted. FIG. 8 provides an isometric view of the sterilized air delivery system 200. The sterilized air delivery system 200 can include a sterilization unit 207; a pair of cooling units 210 with one or more air outputs 220; and an air intake manifold 222 with air inputs 224 into the system (e.g., from an air pump (not shown in this embodiment)). The one or more air outputs 220 can provide the pathogen-free air flow toward, for example, a user mask or another location (e.g., a room or a vehicle cabin). The sterilized air delivery system 200 can be scaled dependent on the output location (e.g., individual user and typically portable; or a room/vehicle cabin and generally fixed in location).

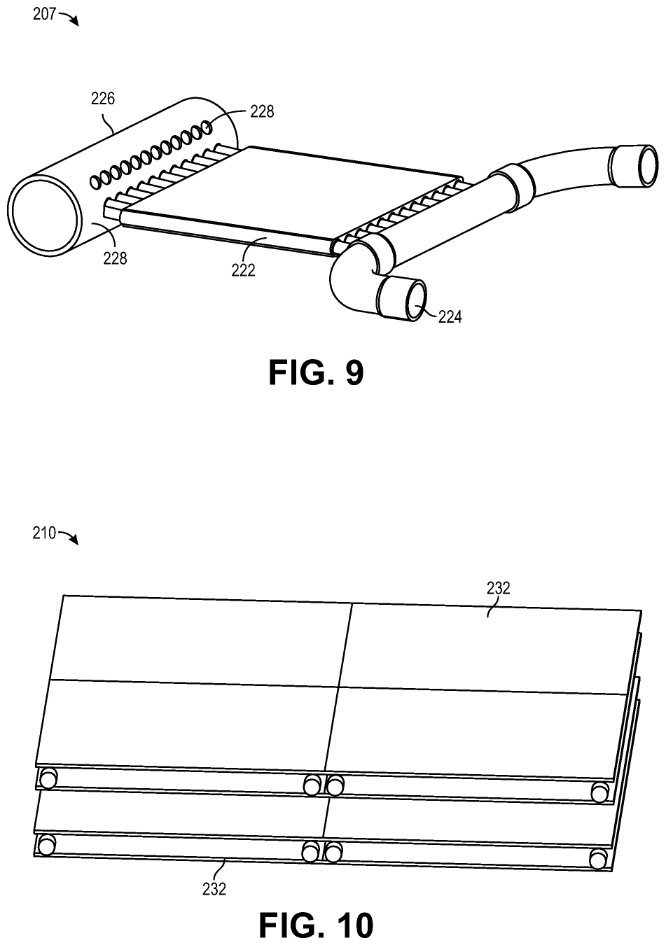

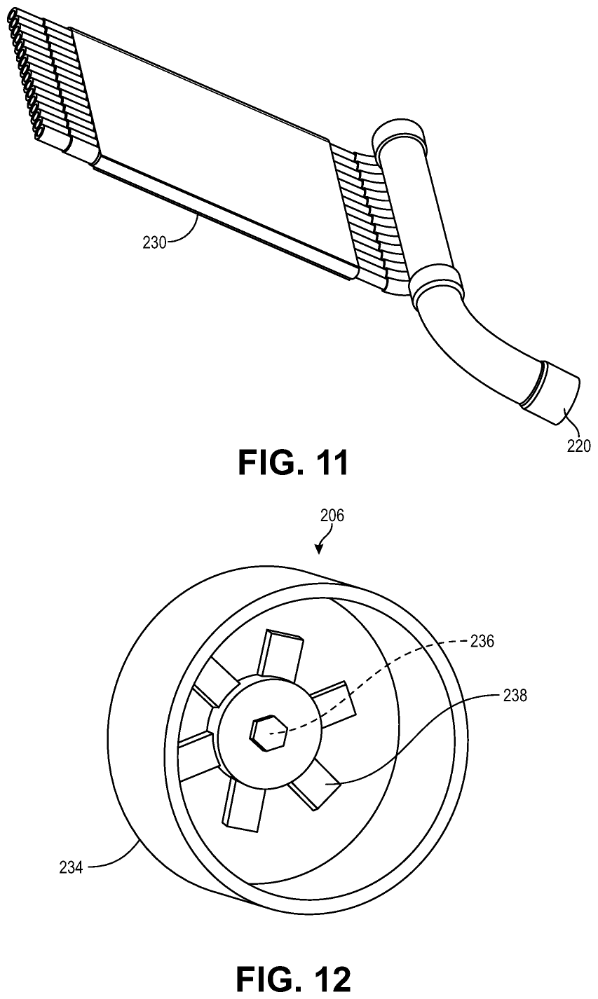

FIG. 9 illustrates the air intake manifold 222 and the air inputs 224, along with the sterilizer unit body 226 of the sterilizer unit 207. The air intake manifold 222 can include one or more intake channels (not labelled). The provision of multiple channels in the air intake manifold 222 can promote flow therethrough and additional heat exchange via the channel walls. Likewise, each cooling manifold 230 can include one or more cooling channels (not labelled). The provision of multiple cooling channels in a given cooling manifold 230 can promote flow therethrough and additional heat exchange via the cooling channel walls. As can be seen in FIG. 9, the sterilizer unit body 226 can define a plurality of body through holes 228 for mating up with the air intake manifold 222 and a respective cooling manifold 230, shown in FIGS. 8 and 11, of each of the cooling units 210, thereby facilitating air flow into and out of the sterilizer unit 207. The forced inflow of air into the sterilizer unit body 226 through the air intake manifold 222 and the cylindrical shape and/or expanded volume of the sterilizer unit 207 can help promote even exposure of the air to UV-C in the sterilizer unit 207, prior to it exiting into a given cooling manifold 230.

In addition to a cooling manifold 230, each cooling unit 210 can include a Peltier heat exchange module 232, as shown in FIGS. 8 and 10, and/or a heat sink (not shown) mounted adjacent to its cooling manifold 230 to facilitate cooling of the sterilized air flowing through a respective cooling manifold 230 and to heat the incoming air within the air intake manifold 222. It is to be understood that the influx of room temperature air through the air intake manifold 222 (e.g., a set or unit of air intake tubes), due to its proximity to the cooling units 210 and the Peltier heat exchange modules 232, can help to cool the air exiting through those cooling units 210, while simultaneously heating the air directed through the air intake manifold 222 and into the sterilizer unit 207. Thus, the air carried by the air intake manifold 222 can act as a heat sink for the cooling units 210. Accordingly, the cooling units 210 can, more broadly, be considered to be a heat exchange unit with a cooling side and a heating side. In an embodiment, a given heat exchange module or unit 232 can be in contact with the air intake manifold 222 and/or a given cooling manifold 230 to promote conductive heat transfer therewith.

In an embodiment, the cooling units 210 (via the hot side of the Peltier heat-transfer module 232) may serve to heat the incoming air in the intake manifold 222. In an embodiment, the heating of the intake air can be sufficient (e.g., to a temperature of 140-150° F. or more; or to a temperature of 200° F. or more) to promote sterilization/killing of any airborne pathogens (e.g., breakdown of lipid coating/shell of virus to make them susceptible to UV radiation; or simply kill a given pathogen). In such an instance, the sterilizer unit body 226 may only carry one or more UV sources (i.e., an additional heat source may be rendered redundant). In an embodiment, the Peltier heat exchange module 232 may simply preheat (e.g., to some temperature above ambient but below the sterilization temperature) the incoming air, with one or more additional heaters (e.g., an IR diode) provided as part of the sterilizer unit 207. Whether the Peltier modules 232 are used in a preheating or heating mode, an advantage of the present system is that the same energy can be utilized for both heating and cooling since a given Peltier module 232 can function as a heat transfer unit transferring heat from one surface thereof to another.

The sterilizer unit body 226 may be insulated or made of a non-thermally conductive material (e.g., a thermally durable plastic; ceramic; etc.) to promote energy usage and/or facilitate handling of the sterilizer unit 207. In some embodiments, the sterilizer unit body 226 may be made of a metal, with or without an internal insulating layer. FIG. 11 shows one of the cooling manifolds 230, along with its corresponding one or more air outputs 220. Both the air intake manifold 222 and a given cooling manifold 230 can be made of a conductive metal such as copper or aluminum to promote conduction of heat therebetween via the Peltier cooling modules 232. In an embodiment, the conductive metal used for such manifolds may further be corrosion resistant. In an embodiment, the UV-irradiated and heated air (i.e., now sterilized air) can be transferred to the cooling manifolds 230 (e.g., sets or units of cooling tubes) located above and below the main intake manifold 222 and their respective Peltier modules 232, using the cool side of a given Peltier module 232 to cool the outgoing air back to ambient temperature or another temperature suitable for breathing, prior to delivery to an end user. Due to the potential for system inefficiencies and/or heat from, for example, the UV LEDs 238, the hot side of each Peltier module 232, and/or any additional heat source, additional Peltier modules 232 and/or heat sinks (not shown) (e.g., on the outer side of each cooling manifold 230, facing away from the intake manifold 222) may be employed to help regulate the return air temperature for comfortable breathing purposes.

The exit air temperature can be measured, for example, by a temperature sensor similar to the optional second temperature sensor 112, and that measured temperature can be used, for example, to control the heat transfer needed by the Peltier modules 232 to bring the outbound air to a suitable temperature (e.g., ambient) for use by an end user. In an embodiment, if the exit air temperature is determined to be outside of an acceptable range (e.g., too hot or too cold), a secondary filtration unit (e.g., N95 mask) or clean air flow (e.g., via a purified air tank) may be enabled (with unacceptable exit temperature air being diverted from end user output) until the cooling unit 210 can again be regulated to bring the exit air within an acceptable exit temperature (neither secondary embodiment shown). Furthermore, a pressure or flow sensor (not shown) may be provided proximate the output of the system 200 and communicatively linked with a control unit (e.g., a computer) to determine if a sufficient flow of sterilized air is being output (e.g., insuring a positive pressure air flow through the system 200). If the output flow or pressure is insufficient, the secondary filtration unit or clean air flow may be temporarily enabled.

FIG. 12 illustrates an end cap 234 of the sterilizer unit 207 for attachment (e.g., by mechanical force fit, screw threading, or metallurgical fastening (e.g., solder or weld)) to a given end of the sterilizer unit body 226. In an embodiment, a pair of end caps 234 can respectively be mounted to opposing ends of the sterilizer unit body 226. In an embodiment, an end cap 234 can form a hermetic seal with a corresponding end of the sterilizer unit body 226. The end cap 234 may be insulated or formed of a non-thermally conductive material, like the sterilizer unit body 226.

In addition to enclosing a given end of the sterilizer unit 207, the end cap 234 can carry at least one of an infrared (IR) diode 236 and/or a UV LED (i.e., light-emitting diode) 238. In an embodiment, the IR diode 236 can be optional, with the necessary heat (e.g., to bring the air to at least 140-150° C.) provided by the hot side of the Peltier heat exchange modules 232. In an embodiment, the IR diode 236 can be configured to heat the air within the sterilizer unit 207 to at least 140-150° C. or another temperature as needed to promote killing of pathogens within the air (e.g., upon being preheated to some level by the Peltier heat exchange modules 232). The UV LED 238 can promote killing of any airborne pathogens within the sterilization unit 207 before the air passes to a given cooling manifold 230 of a corresponding cooling unit 210. In an embodiment, the UV LED 238 can be effective in killing viruses, particularly if heat has been used to first breakdown any lipid coating/shell surrounding a given virus. The end cap 234 and the UV LED 238 may together be considered to define a UV unit 206. In an embodiment, the UV LED 238 can generate an amount of heat, thus helping to heat the air within the sterilizer unit 207. In an embodiment, the end cap 234 may carry a heat sink (e.g., heat dissipating fins) to help maintain the UV unit 206 at a desirable working temperature.

In an embodiment, two or more UV LEDs 238 can be provided (e.g., in a given end cap 234). In an embodiment, the two or more UV LEDs 238 can respectively be configured to generate different wavelengths of UV radiation, each capable of targeting a different type of virus or other pathogen. In an embodiment, one or more UV LEDs 238 can be provided in locations other than at the end caps 234 (e.g., the sterilizer unit body 226, the air intake manifold 222, and/or the respective cooling manifolds 230—not shown in such locations) to promote greater exposure of the air processed by the sterilized air delivery system 200 to one or more wavelengths of UV radiation. For example, the UV LEDs 238 can be positioned at various locations throughout the system 200 to dose the processed air with UV radiation throughout its dwell time within the system 200.

It is to be understood that a given sterilizer unit 207 may employ at least one IR diode 236 or at least one UV LED 238, but not necessarily both. That is, in embodiments, a sterilizer unit 207 may sterilize the air with heat and/or UV radiation. Further, a sterilization unit 207 may use the heat transferred from the Peltier modules 232 and/or heat generated by the one or more UV LED's 238 as the heat source to reach a sterilization temperature, obviating the need for any IR diodes 236 or another additional heat source. In an embodiment, both ends of the sterilizer unit body 226 may include an end cap 234 to contain the air processed within the sterilizer unit 207 (e.g., the end caps 234 and the sterilizer unit body 226 together forming a hermetically sealed unit in fluid communication with the inbound and outbound manifolds). In an embodiment, each end cap 234 can be provided with at least one UV LED 238 to ensure a sufficient supply of UV-C irradiation to promote neutralization of any viruses or other airborne pathogens.

In an embodiment, the sterilizer unit body 226 and the end caps 234 of the sterilizer unit 207 may together form a tank 239. The tank 239 (e.g., due to its cylindrical or near-cylindrical shape and/or expanded volume relative to the air intake manifold 222 and the respective cooling manifolds 230) can help disrupt the air and otherwise increase the turbulence thereof to mix it up and separate the larger particles suspended in the relative humidity. In tests, it has been found that the relative humidity (RH) can significantly affect the success of viral termination. As the heat and/or available volume promote expansion of the air, the RH decreases, and the outer shield on the virus can be more readily stripped, therefore letting the UV act on the virus quicker. The tank 239 can also provide a greater volume that can be needed to allow time in UV light to work if the user is breathing hard (i.e., level of breathing can affect the dwell time of the air within the system; and a minimum dwell time can be needed for the heat and/or UV to sterilize the processed air). In an embodiment, the tank 239 can be a shape other than cylindrical (not shown), so long as it provides an expanded volume (relative to the air intake manifold 222 and the respective cooling manifolds 230) for air flow disruption/mixing and expansion and creates a flow path from the air intake manifold 222 toward the one or more cooling manifolds 230.

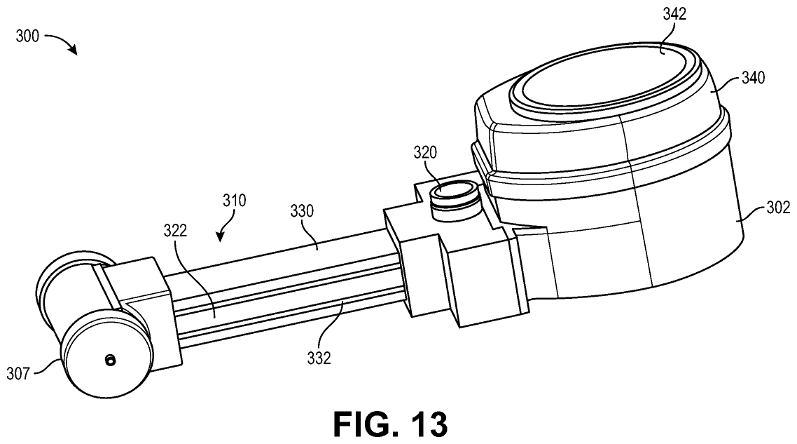

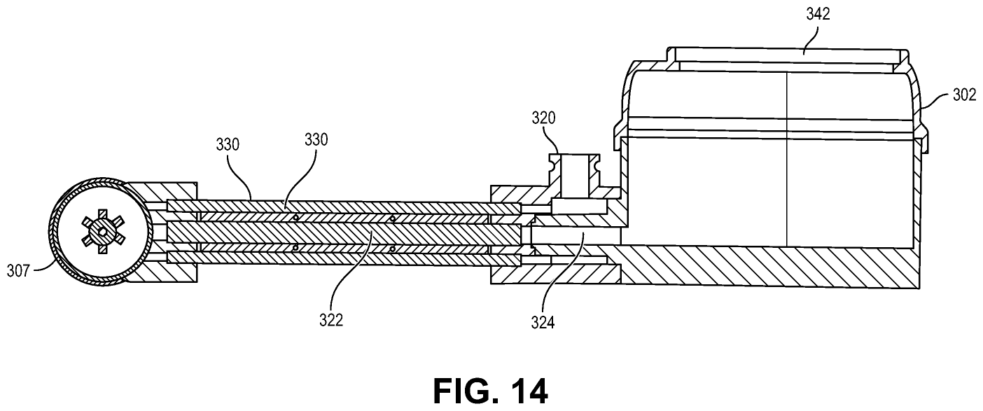

FIGS. 13 and 14 illustrate a sterilized air delivery system 300, in accordance with an example embodiment of the present disclosure. The sterilized air delivery system 300 is similar to the sterilized air delivery system 200, except that it expressly incorporates an air pump 302. As such, similar numbered parts for the sterilized air delivery systems 200 and 300 can be expected to be constructed and to operate in a similar manner. The sterilized air delivery system 300 can include the air pump 302, a sterilizer unit 307, a cooling unit 310, and an air intake manifold 322, in fluid communication with one another. The sterilizer unit 307, like the sterilizer unit 207, can be equipped to sterilize the air flow therethrough using heat and/or UV radiation (e.g., via an IR diode and/or a UV LED). The cooling unit 310 can include at least one cooling manifold 330 and at least one Peltier heat exchange module 332 or a heat sink (not shown). A given cooling manifold 330 can include at least one air output 320 for directing the sterilized (i.e., pathogen-free) air, via a fluid connection (e.g., conduit, tubing, or piping), to, for example, a face mask or a room (neither shown). The air intake manifold 322 can include at least one air input 324 for fluid interconnection with the air pump 302. The air pump 302 can include a pump housing 340, an entry filter 342 (e.g., for removing particulates such as dust from the air), and a pump fan (not shown).

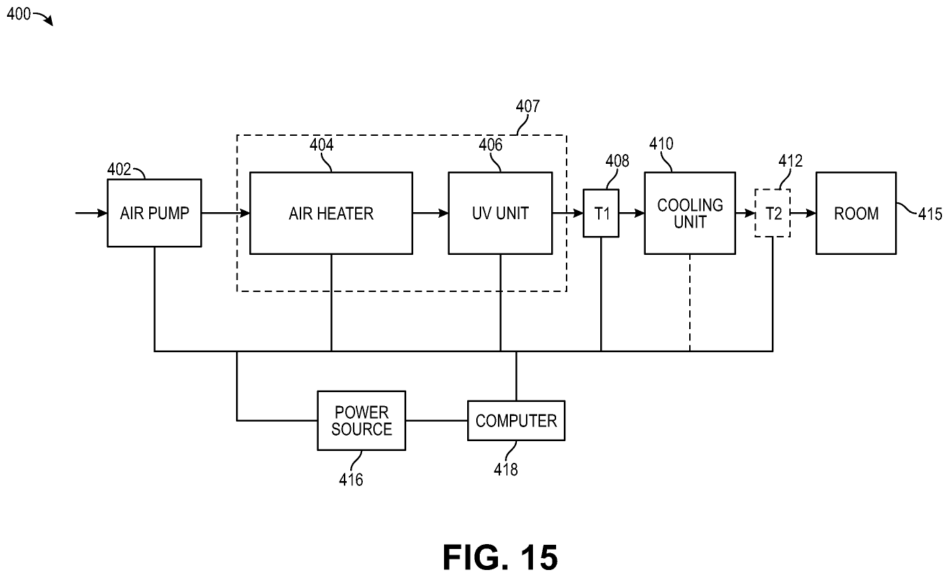

FIG. 15 illustrates a room air delivery system 400, in accordance with an example embodiment of the present disclosure. The room air delivery system 400 can generally include an air pump 402, an air heater 404 (e.g., a Peltier heat exchange module which can serve to heat incoming air and cool exiting air; and/or an IR diode), a UV unit 406, a first temperature sensor 408 (i.e., to confirm sufficient heating (e.g., 140° F. or greater)), a cooling unit 410, an optional second temperature sensor 412 (e.g., to confirm sufficient cooling for user breathing (e.g., to room temperature)), a room or cabin 415 (e.g., a room of a building, tent, etc., or a cabin of a vehicle), a power source 416, and a computer 418. In some embodiments, the room air delivery system 400 can use heat and/or UV radiation to sterilize the air, and a single sterilizer unit 407 may be used to house one or both of an air heater 404 or a UV unit 406. Similar numbered parts as with the wearable air delivery system 100 and the sterilized air delivery system 200 can be expected to be constructed and function in a similar manner (e.g., size may, however, vary to accommodate the load and/or space requirements on the system), unless otherwise noted.

It is to be understood that the room air delivery system 400 may be able to deliver pathogen-free (i.e., sterilized) air to one or more rooms. The user mask 114 and/or the room or cabin 415 effectively can serve as units for receiving and outputting the pathogen-free (i.e., sterilized) air flow to one or more end users of that air. In some embodiments, the room air delivery system 400 may be used to sterilize air incoming to a room or cabin 415 or air exiting a room or a cabin 415 (e.g., for exhaust to the environment or to another location in a building). If used to exhaust pathogen-free air to the environment, the air processed thereby may not necessarily have to be cooled to a level fit for immediate human or animal consumption. The room air delivery system 400 may be part of a negative, atmospheric, or positive pressure system for a given room or cabin 415 and may be used in conjunction with one or more other room air delivery systems 400 (e.g., different systems dedicated to treating either incoming or exiting air). In an embodiment, the level of inbound and/or outbound airflow created by the one or more systems 400 can be adjusted to create a desired pressure (e.g., positive, atmospheric, or negative) within the room or cabin 415.

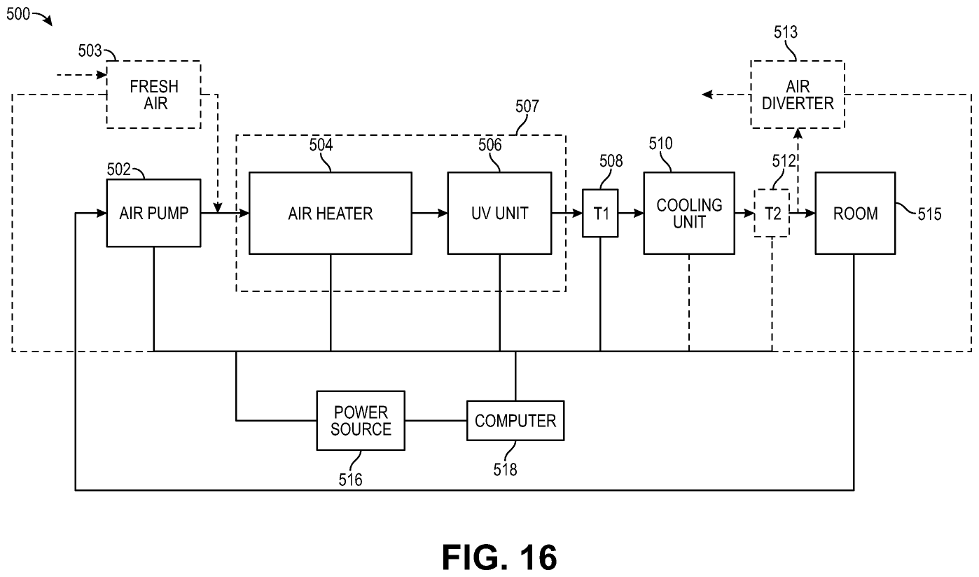

FIG. 16 shows a schematic view of a recirculating room air delivery system 500, in accordance with an embodiment of the present disclosure. The room air delivery system 500 can generally include an air pump 502, an optional air intake unit 503, an air heater 504, a UV unit 506, a first temperature sensor 508 (i.e., to confirm sufficient heating (e.g., 140° F. or greater)), a cooling unit 510, an optional second temperature sensor 512 (e.g., to confirm sufficient cooling for user breathing (e.g., to room temperature)), an optional air diverter unit 513 a room or cabin 515 (e.g., a room of a building, tent, etc., or a cabin of a vehicle), a power source 516, and a computer 518. The air heater 504 and the UV unit 506 may be part of a sterilizer unit 507. The recirculating room air delivery system 500 is similar in operation and construction to the room delivery air system 400 (i.e., similar numbered parts can be expected to be constructed and function in a similar manner) except, in part, that the recirculating room air delivery system 500 is configured to draw air from the room or cabin 515 (i.e., fluidly connected thereto). Thus, the recirculating room air delivery system 500 can sterilize the air before introducing all or a portion of it back into the room or cabin 515.

The recirculating room air delivery system 500 can include the optional air intake unit 503, which can be controlled by the computer 518 to facilitate intake of fresh air into the system 500 and ultimately into the room or cabin 515. The optional air intake unit 503 can include, for example, an intake pump and/or a control valve (neither shown) to regulate the intake of the fresh air. The recirculating room air delivery system 500 may be controlled to create a positive, an atmospheric, or a negative pressure in the room or cabin 515.

In an embodiment, the optional air diverter unit 513 can be used to create a negative pressure in the room or cabin 515 (e.g., to help avoid cross-contamination from room to room, for example, in a hospital or other medical facility). In an embodiment, the optional air diverter unit 513 can include a diverter control valve and/or a diverter exhaust fan (not shown) to draw a portion of the overall air flow from the cooling unit outflow and divert it away (e.g., to the environment or another room in a building) from the room or cabin 515. In an embodiment, such elements as the diverter control valve and/or the diverter exhaust fan can be controlled via the computer 518. In an embodiment, the optional air diverter unit 513 may rely simply on plumbing features (e.g., wider conduit to outflow than to room; or manual flow valves) to achieve the desired negative pressure.

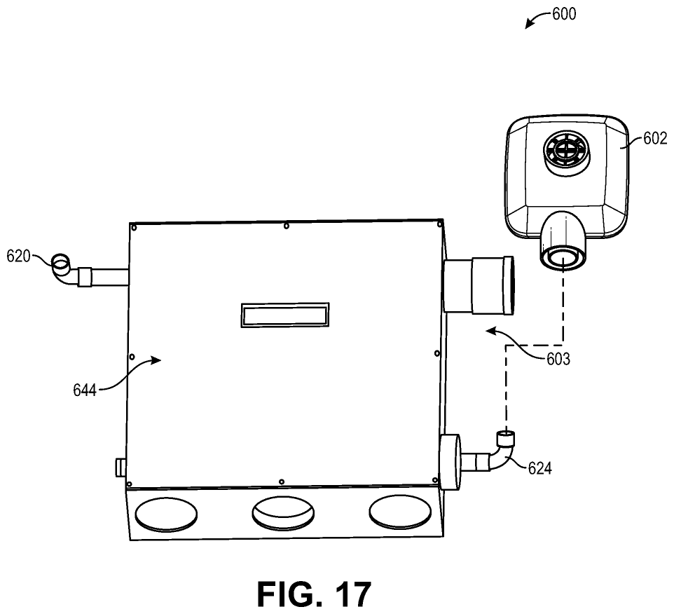

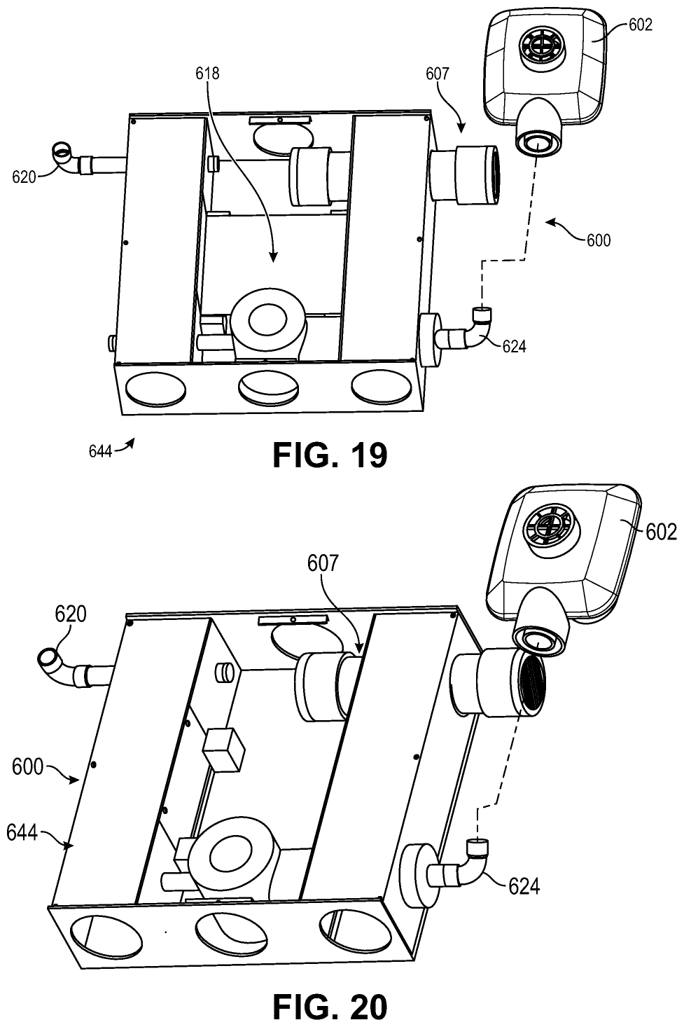

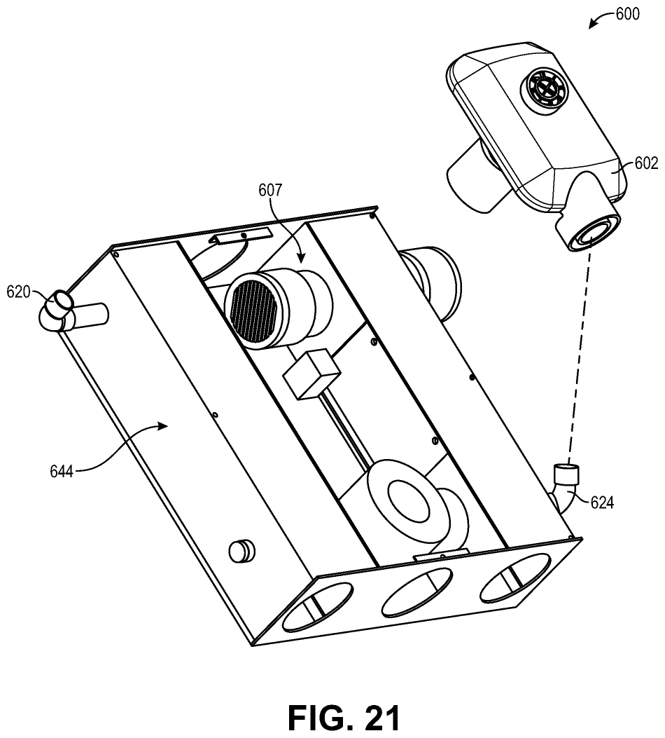

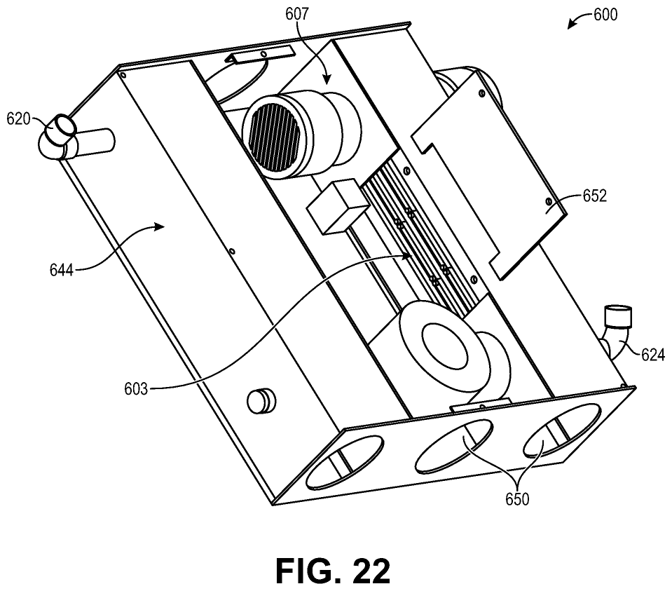

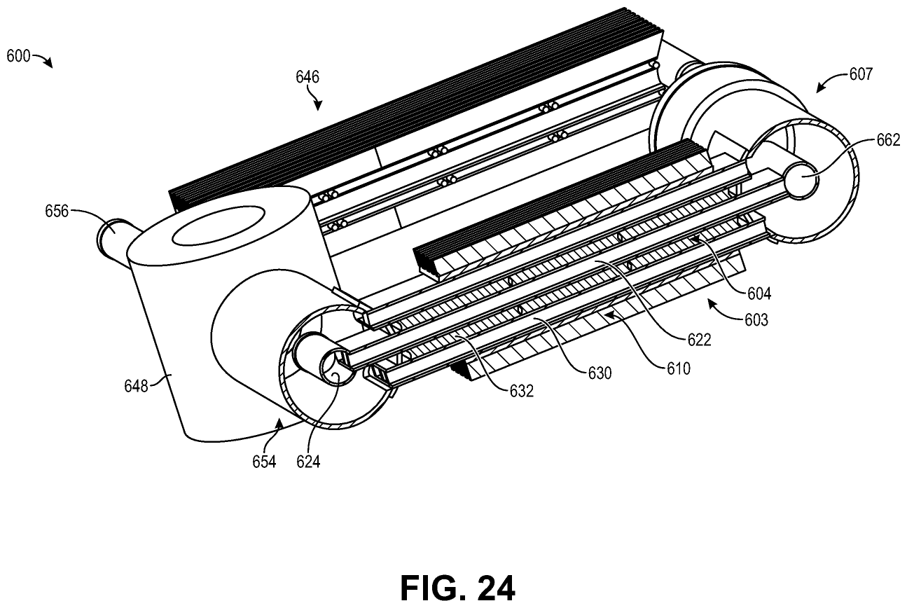

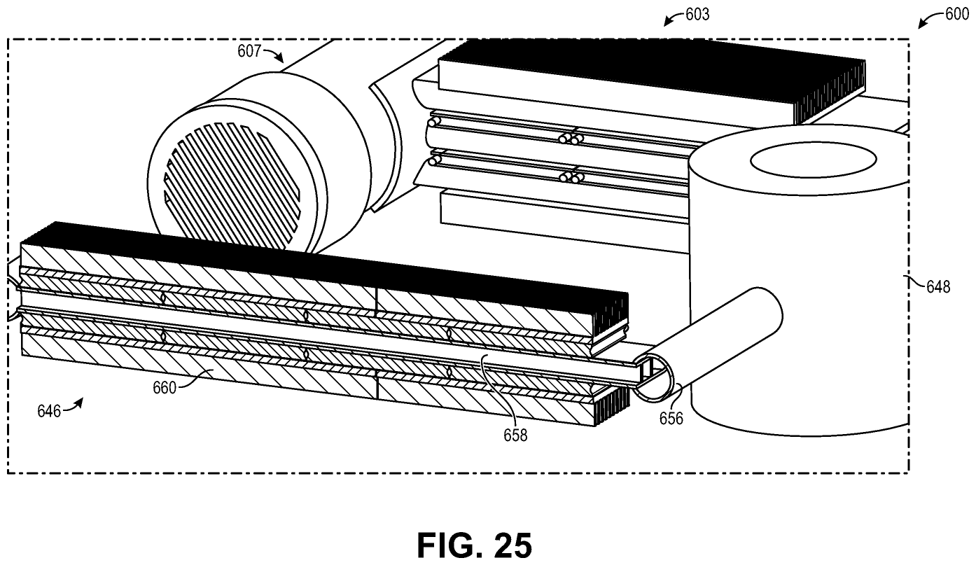

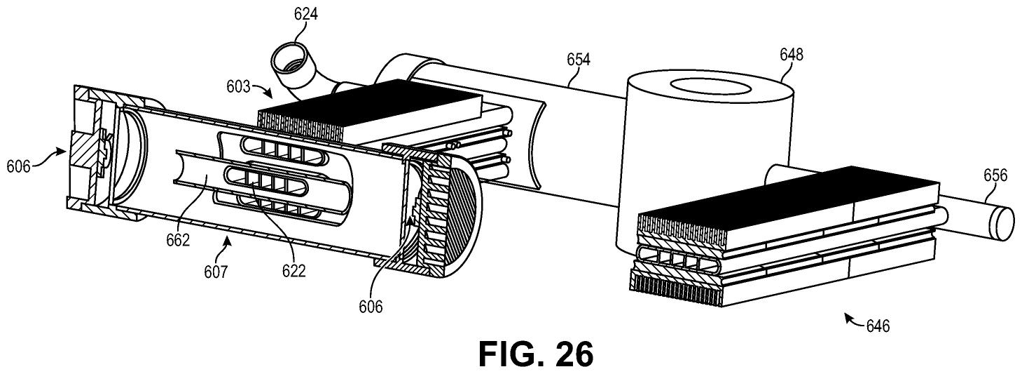

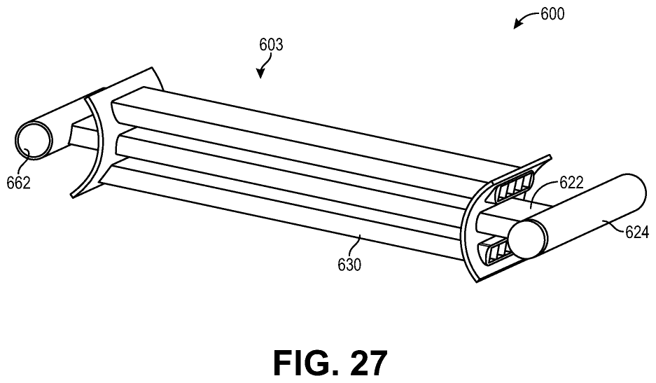

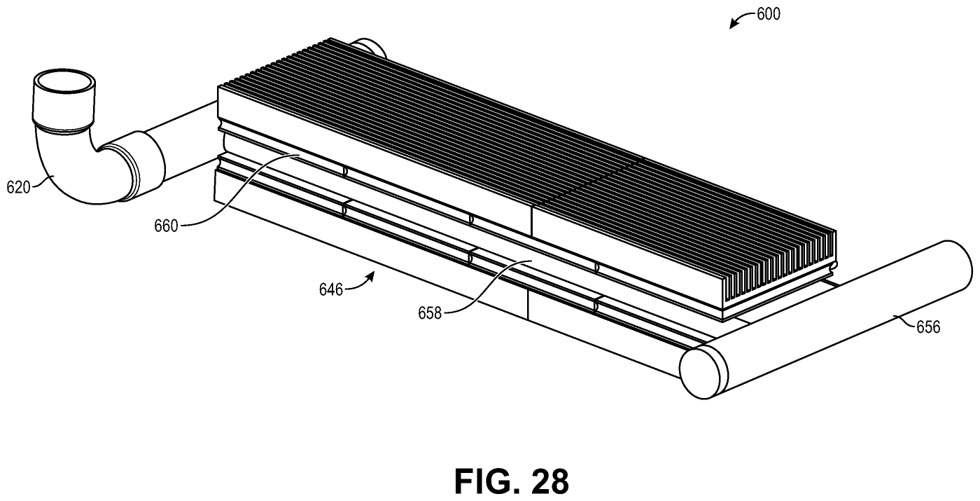

FIGS. 17-29 together show a sterilized air delivery system 600, in accordance with an embodiment of the present disclosure. The sterilized air delivery system 600 is similar in operation and construction to the other sterilized air delivery systems, particularly 100, 200, 300, so similar numbered parts can be expected to be constructed and function in a similar manner. However, the sterilized air delivery system 600 differs from other embodiments, in part, in that it provides a secondary cooling unit 646 and a system housing 644. It is to be understood that elements (e.g., the secondary cooling unit 646) of the sterilized air delivery system 600 may be used in conjunction with other embodiments, and such usage is considered within the scope of the present disclosure.

The sterilized air delivery system 600 can include an air pump 602, a primary heat exchanger unit 603 (e.g., a heat pump) including an air heater 604 and a pair of cooling units 610, at least one UV unit 606 (e.g., a pair), a sterilizer unit 607, an on-board computer or processor 618, an air output 620, an air intake manifold 622 (i.e., part of the air heater 604), an air input 624, a pair of cooling manifolds 630, a pair of cooling units 631 (e.g., a Peltier cooling unit and/or a heat sink (e.g., cooling fins) mounted exterior of a given cooling manifold 630), and a pair of Peltier cooling units 632 (e.g., between a given cooling manifold 630 and the air intake manifold 622). The sterilized air delivery system 600 can further include a system housing 644, a secondary cooling system 646 including a secondary cooling manifold 658 and at least one secondary cooling unit 660 (e.g., a Peltier cooling unit and/or a heat sink (e.g., cooling fins)). The air exiting the pair of cooling manifolds 630 can be fluidly connected to the secondary cooling manifold 658 of the secondary cooling system 646, for example, by a transitional fluid interconnect 648, a first cooling interconnect (e.g., tubing/conduit) 654, and a second cooling interconnect (e.g., tubing/conduit) 656. The system housing 644 can define one or more removable housing sections 652 and/or one or more housing ports 650 (e.g., to facilitate access into the system housing 644 and/or to promote airflow around the components housed thereby). The sterilized air delivery system 600 can further include a redirecting tube 662 fluidly connected to an exit end of the air intake manifold 622 and positioned within the sterilizer unit 607 in a manner to direct air flow toward a given UV unit 606 (e.g., two distal open ends, each proximate a corresponding UV unit 606).

As seen from FIGS. 17-22, the system housing 644 can enclose and carry, for example, the primary heat exchanger 603, the sterilizer unit 607 housing at least one given UV unit 606, the secondary cooling unit 646, and any fluid interconnects (e.g., 648, 654, 656) therebetween. The system housing 644 may further enclose and/or carry an on-board computer 618 and/or at least one battery unit or power connection (latter elements not illustrated). In an embodiment, the walls of the system housing 644 may be thermally conductive or thermally non-conductive, as desired. For example, the outer walls of the system housing 644 may be thermally conductive to help with the cooling of the overall unit 600. In some embodiments, at least one of the interior walls of the system housing 644 may be insulated or made of a thermally nonconductive (e.g., made of a ceramic or a thermoplastic) to limit outside heat transfer from the primary heat exchanger 603 to the secondary cooling system 646.

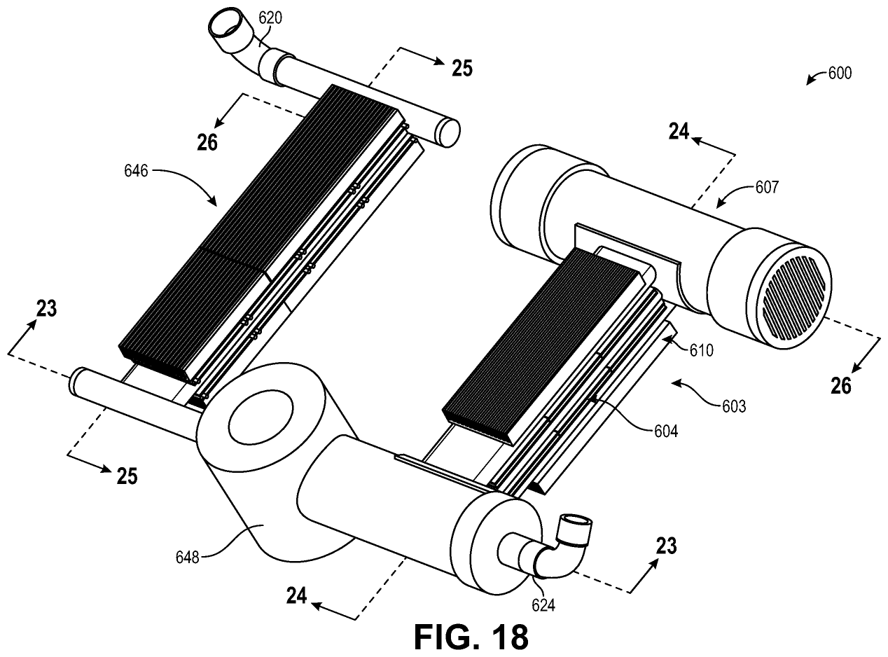

In embodiments, the primary heat exchanger 603, as illustrated in FIGS. 18 and 23-27, can be configured to heat air incoming through the air intake manifold 622 and simultaneously cool air travelling through the one or more cooling manifolds 630 via the Peltier cooling/heat exchange units 632. The Peltier cooling units 632 can act to pump heat from the outbound air being cooled to the inbound air being heated (i.e., the inbound air can act as a heat sink for the air being cooled). In an embodiment, the heat pump action of the one or more Peltier cooling/heat exchange units 632 can raise the temperature of the incoming air to a temperature of 140° F. or more, or even 200° F. or more.

In an embodiment, the first cooling interconnect 654 can be a conduit fluidly interconnected to the pair of cooling manifolds 630 (e.g., via an opening in a sidewall of the first cooling interconnect 654). The first cooling interconnect 654 can also separately house a portion of the air intake 624, with the air intake fluidly interconnected with the air intake manifold 622. In an embodiment, the first cooling interconnect 654 can be fluidly connected to the second cooling interconnect 656 via the transitional fluid interconnect 648. The transitional fluid interconnect 648 can be configured to fluidly connect a larger diameter tube (e.g., 654) to a smaller diameter tube (e.g., 656. In an embodiment, the transitional fluid interconnect 648 may provide a transitional internal diameter change (e.g., sloped/frustoconical) to improve the flow between the different diameter components. In some embodiments, the transitional fluid interconnect 648 may be provided with a UV source (e.g., a UV LED, not shown in this location) to create another position at which the airflow may be exposed to UV-C radiation.

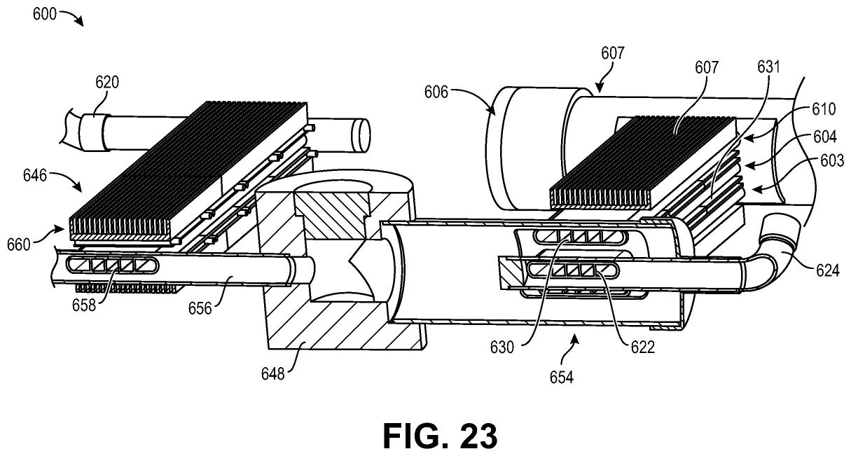

In an embodiment, the secondary cooling system 646, as seen, for example, in FIGS. 18 and 23, can be fluidly connected between the exit from the cooling manifolds 630 of the primary heat exchanger unit 603 and the air output 620. In some embodiments, the secondary cooling system 646 may be spaced and/or insulated from the primary heat exchanger 603. Such spacing or insulating can help maximize the cooling capability of the secondary cooling system 646 (e.g., use cooling capability to cool air flowing therethrough while minimizing cooling needed for external heat sources). It is to be understood that the relative position between the secondary cooling system 646 and the primary heat exchanger 603 and the fluid connection(s) therebetween may be adjusted, for example, based on space considerations and/or heat exchange requirements. In an embodiment where the distance therebetween is reduced, a system housing 644 with insulated and/or thermally nonconductive interior/separating walls (e.g., ceramic and/or thermoplastic) may be employed.

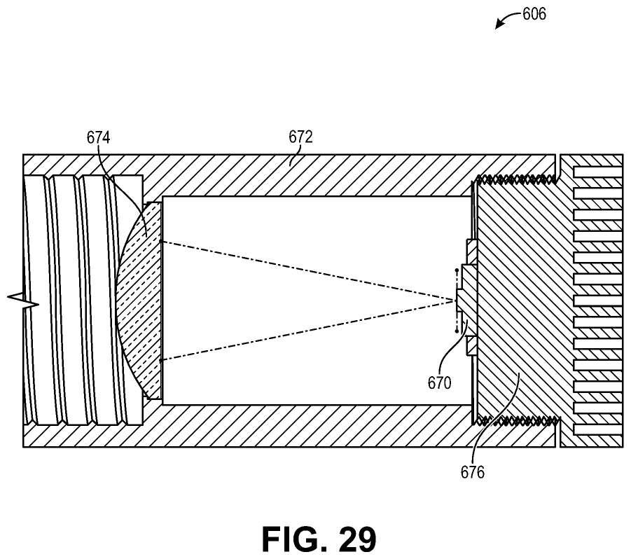

In some embodiments, the UV unit 606, as shown in FIG. 29, can include a UV LED 670, a dual threaded-end unit housing 672, a focus lens 674, and a heat-sink end cap 676. A first end of the dual threaded-end unit housing 672 may be able to screw onto a corresponding end of the sterilizer unit 607, making an air-tight connection therebetween. The second end of the dual threaded-end unit housing 672 may threadedly connect with the heat-sink end cap 676, making an air-tight seal between those components. The heat-sink end cap 676 can be configured to carry the UV LED 670 on an interior face thereof. In an embodiment, the UV LED 670 may be releasably attached (e.g., snap-fit; screw threading; etc.) to the heat-sink end cap 676 to facilitate changing thereof when needed. The heat-sink end cap 676 can define heat-sink cooling fins on an exterior face thereof, to promote cooling of the UV LED 670. The focus lens 674 can be carried within the dual threaded-end unit housing 672 at distance away from the UV LED 670 to promote focusing of UV light emitted from the UV LED 670 (e.g., focused toward an opening end of the redirecting tube 662).

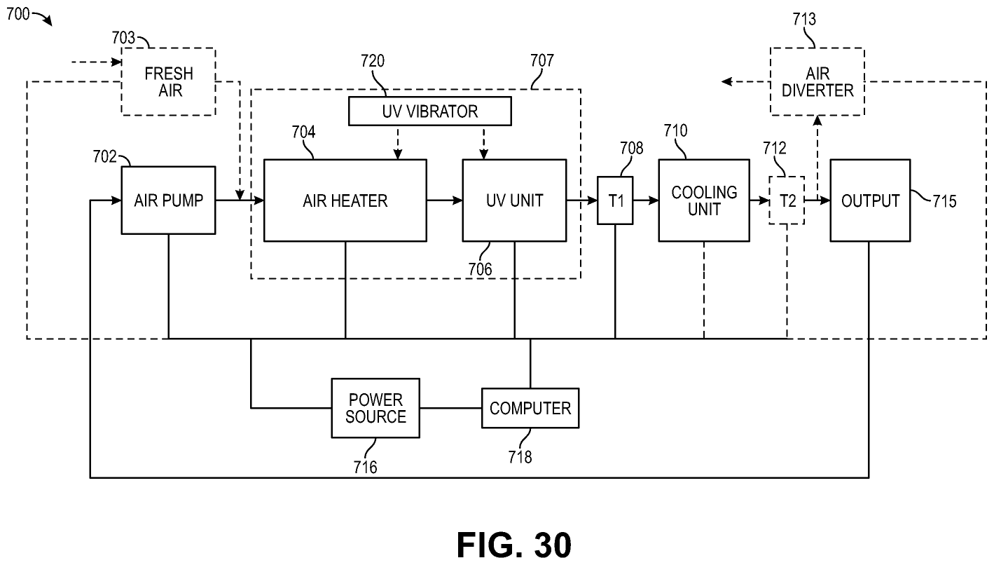

FIG. 30 illustrates a schematic view of a sterilized air delivery system 700, in accordance with an embodiment of the present disclosure. The sterilized air delivery system 700 can generally include an air pump 702, an optional air intake unit 703, an air heater 704, a UV unit 706, a first temperature sensor 708 (i.e., to confirm sufficient heating (e.g., 140° F. or greater)), a cooling unit 710, an optional second temperature sensor 712 (e.g., to confirm sufficient cooling for user breathing (e.g., to room temperature)), an optional air diverter unit 713, an output 515 (e.g., a room of a building, tent, etc., or a cabin of a vehicle; or a user mask), a power source 716, a computer 718 (e.g., a microcontroller, a personal computer, or network system), and an ultrasonic (US) vibrator 720. The air heater 704, the UV unit 706, and/or the ultrasonic vibrator 720 may be part of a sterilizer unit 707. The sterilized air delivery system 700 can be similar in operation and construction to the air delivery systems 400 and/or 500 (i.e., similar numbered parts can be expected to be constructed and function in a similar manner).

The sterilized air delivery system 700 differs, at least in part, from the air delivery systems 400 and/or 500 in that it can include an ultrasonic vibrator 720. In an embodiment, the ultrasonic vibrator 720 may include an ultrasound generating transducer. The ultrasonic vibrator 720 may, for example, be part of the sterilizer unit 707 and may be configured to ultrasonically vibrate the air flow through the sterilizer unit 707 when that air flow is heated and/or when the air flow is subjected to UV radiation. The ultrasonic vibrations may aid in the disruption of the capsid (e.g., fatty layer) protecting the virus or other microbe and/or promote the destruction thereof. In an embodiment, the sonic and/or ultrasonic vibrations can be directed within a retention tank (not shown) associated with the sterilizer unit 707. It is to be understood that any of the sterilized air delivery systems 100-600 may be modified to incorporate a corresponding ultrasonic vibrator and be within the scope of the present disclosure.

In an embodiment, the sterilized air delivery system 700 may use the air pump 702 to selectively generate negative (e.g., vacuum level) or positive pressure flows and/or to create rapidly changing pressures. For example, rapidly changing pressures within the system (particularly within the sterilizer unit 707) may help disrupt the capsid protecting the virus, making it more susceptible to heat and/or UV radiation. It is to be understood that any combination of heat, UV radiation (e.g., UV-C), pressure (including fluctuating pressure), or ultrasonic vibrations to destroy a given virus or other microbe is considered to be within the scope of the present system. In an embodiment, the combination of heat, UV radiation, pressure, or ultrasonic vibrations that yields virus and/or microbe inactivation with the shortest dwell time and/or the lowest energy output may be chosen. In an embodiment, that combination is chosen such that breathable air is generated without having to hold the air for batch treatment, instead allowing for continuous airflow therethrough. It is to be understood that any of the sterilized air delivery systems 100-600 may be modified to incorporate an air pump that allows for variable pressures (e.g., positive, negative, and/or rapid variance) and be within the scope of the present disclosure.

In an embodiment, the humidity may be controlled in any of the sterilized air delivery systems 100-700. For example, the capsid (e.g., fatty layer) on a virus may have an optimal humidity range, above which or below which its integrity may be adversely affected. As such, in an embodiment, the humidity may be controlled to be outside of the optimal range for the capsid and thus help promote the breakdown of the capsid layer of a given virus.

With any of the sterilized air delivery systems 100-700, the goal is to yield breathable air (e.g., appropriate temperature and chemical make-up), free of any living viruses and/or microbes. In an embodiment, the incidental killing of other living organisms in that air may not be a concern. In an embodiment, the process of creating that breathable air does not increase the ozone level therein and/or does not breakdown any water vapor therein into oxygen and hydrogen. In an embodiment, a given sterilized air delivery systems 100-700 does not require a licensed technician for its use and/or operation.

The sterilized air delivery systems 100, 200, 300, 400, 500, 600, and 700, including some or all of its components, can operate under computer control, such as a given computer 118, 418, 518, 618, or 718. The computer can be in the form, for example, of a separate computer unit (e.g., laptop, mainframe, tablet, single-board computer (such as a Raspberry Pi computer), etc.) in a wired or wireless connection with a given air delivery system 100-700 or can be an embedded computer (e.g., a programmable logic controller or a miniature computer unit) built within a given system 100-700. A given computer (e.g., 118, 418, 518, 618, or 718) can include a processor (not shown) to control the components and functions of a given air delivery system 100-500 described herein using software, firmware, hardware (e.g., fixed logic circuitry), manual processing, or a combination thereof. The terms “controller,” “functionality,” “service,” and “logic” as used herein generally represent software, firmware, hardware, or a combination of software, firmware, or hardware in conjunction with controlling a given air delivery system 100-700. In the case of a software implementation, the module, functionality, or logic represents program code that performs specified tasks when executed on a processor (e.g., central processing unit (CPU) or CPUs). The program code can be stored in one or more computer-readable memory devices (e.g., internal memory and/or one or more tangible media), and so on. The structures, functions, approaches, and techniques described herein can be implemented on a variety of commercial computing platforms having a variety of processors.

A processor can provide processing functionality for a given air delivery system 100-700 and can include any number of processors, micro-controllers, or other processing systems, and resident or external memory for storing data and other information accessed or generated by a given air delivery system 100-700. The processor can execute one or more software programs that implement techniques described herein. The processor is not limited by the materials from which it is formed or the processing mechanisms employed therein and, as such, can be implemented via semiconductor(s) and/or transistors (e.g., using electronic integrated circuit (IC) components), and so forth.

The computer (e.g., 118, 418, 518, 618, or 718) for a given air delivery system 100-700, as needed, can also include a memory (not shown). The memory is an example of tangible, computer-readable storage medium that provides storage functionality to store various data associated with operation of a given air delivery system 100-700, such as software programs and/or code segments, or other data to instruct the processor, and possibly other components of a given air delivery system 100-700, to perform the functionality described herein. Thus, the memory can store data, such as a program of instructions for operating a given air delivery system 100-700 (including its components), and so forth. It is noted that while a single memory is described, a wide variety of types and combinations of memory (e.g., tangible, non-transitory memory) can be employed. The memory can be integral with the processor, can comprise stand-alone memory, or can be a combination of both. The memory can include, but is not necessarily limited to: removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In implementations, the memory can include removable integrated circuit card (ICC) memory, such as memory provided by a subscriber identity module (SIM) card, a universal subscriber identity module (USIM) card, a universal integrated circuit card (UICC), and so on.

The computer for a given air delivery system 100-700 can further include a communications interface (not shown). The communications interface is operatively configured to communicate with components of the system. For example, the communications interface can be configured to transmit data for storage in the system, retrieve data from storage in the system, and so forth. The communications interface is also communicatively coupled with the processor to facilitate data transfer between components of the system and the processor (e.g., for communicating inputs to the processor received from a device communicatively coupled with the system and/or communicating output to a device communicatively coupled with the system. It is noted that while the communications interface is described as a component of a system, one or more components of the communications interface can be implemented as external components communicatively coupled to the system via a wired and/or wireless connection. The system can also comprise and/or connect to one or more input/output (I/O) devices (e.g., via the communications interface) including, but not necessarily limited to: a display, a mouse, a printer or printer/scanner, and so on. The communications interface and/or the processor can be configured to communicate with a variety of different networks (e.g., wireless and/or wired).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An air delivery system for delivering a pathogen-free airflow, the air delivery system comprising:
an air intake manifold for receiving an incoming air flow;
a sterilizer unit in fluid communication with the air intake manifold, the sterilizer unit comprising a tank and at least one ultraviolet (UV) unit mounted within the tank, the at least one ultraviolet unit configured to generate ultraviolet light sufficient to promote killing of airborne pathogens;
a cooling unit positioned after the sterilizer unit and in fluid communication therewith, the cooling unit comprising at least one cooling manifold and at least one heat exchange unit, the at least one heat exchange unit positioned and configured to cool the outbound air flow to a temperature suitable for breathing by a user and to heat the incoming air flow in the air intake manifold; and
a user output in fluid communication with the cooling manifold, the user output configured to deliver the cooled outbound air flow to an end user, the cooled air flow being pathogen-free.

2. The air delivery system of claim 1, wherein the user output is one of an air mask, a room, or a vehicle cabin.

3. The air delivery system of claim 1, wherein the incoming air flow is heated by at least one heat exchange unit to a temperature sufficient for at least one of killing or breaking down a lipid shell of the airborne pathogens.

4. The air delivery system of claim 3, wherein at least one given heat exchange unit comprises a Peltier heat exchange unit.

5. The air delivery system of claim 4, further comprising a first temperature sensor configured to confirm heating of the air flow at least within or exiting the sterilizer unit to a temperature sufficient to at least one of promote a breakdown of a lipid shell of the airborne pathogens or kill the airborne pathogens.

6. The air delivery system of claim 1, wherein each heat exchange unit is configured to have a cooling side and a heating side, the cooling side of a given heat exchange unit configured to cool the outbound air flow in a given cooling manifold, the heating side of the given heat exchange module positioned proximate the air intake manifold, the heating side of the given heat exchange module configured to heat the incoming air flow in the air intake manifold.

7. The air delivery system of claim 6, the at least one heat exchange unit is configured to generate enough heat to raise the temperature in the air intake manifold to a temperature sufficient to at least one of kill a given pathogen or break down a lipid shell thereof.

8. The air delivery system of claim 1, wherein the sterilizer unit further comprises an ultrasonic vibrator.

9. The air delivery system of claim 1, further comprising at least one of a pump or fan in fluid communication with the air intake manifold, the at least one of a pump or fan configured to variably adjust a pressure of the air flow in the sterilizer unit.

10. The air delivery system of claim 1, further comprising at least one heating unit mounted in the sterilizer unit, the at least one heating unit configured to raise the temperature of the air in the sterilizer unit to a temperature sufficient to at least one of kill a given pathogen or break down a lipid shell thereof.

11. The air delivery system of claim 1, further comprising a secondary cooling system located between the cooling unit and the user output, the secondary cooling system comprising at least one of a Peltier heat exchange module or a heat sink.

12. The air delivery system of claim 1, wherein the at least one UV unit comprises at least one UV LED.

13. The air delivery system of claim 1, wherein the at least one UV unit comprises a plurality of UV units, at least two given UV units configured to generate different respective wavelengths of UV radiation.

14. The air delivery system of claim 1, wherein the at least one UV unit is configured to generate at least one wavelength in a UV-C range.

15. An air delivery system for delivering a pathogen-free airflow, the air delivery system comprising:
an air intake manifold for receiving an incoming air flow;
a cooling unit in fluid communication therewith, the cooling unit comprising at least one cooling manifold and at least one heat exchange unit, the at least one heat exchange unit positioned and configured to cool the outbound air flow to a temperature suitable for breathing by a user and to heat the incoming air flow in the air intake manifold, the air intake manifold and the at least one cooling manifold comprising an air flow path of the air delivery system;
at least one ultraviolet (UV) unit mounted in the air flow path of the air delivery system, the at least one UV unit configured to generate ultraviolet light sufficient to promote killing of pathogens; and
a user output in fluid communication with the cooling manifold, the user output configured to deliver the cooled outbound air flow to an end user, the cooled air flow being pathogen-free.

16. The air delivery system of claim 15, wherein at least one UV unit is mounted at a position between the air intake manifold and the at least one cooling manifold.

17. The air delivery system of claim further comprising a tank in fluid communication between the air intake manifold and the at least one cooling manifold, at least one given UV unit mounted in the tank.

18. The air delivery system of claim 15, wherein the user output is one of an air mask, a room, or a vehicle cabin.

19. The air delivery system of claim 15, wherein the at least one heat exchange unit is configured to generate enough heat to raise the temperature in the air intake manifold to at least 140° F.

20. The air delivery system of claim 15, wherein at least one given heat exchange unit comprises a Peltier heat exchanger.
